(19) United States
(12) Patent Application Publication
(10) Pub. No.: US 2004/0092588 A1
(43) Pub. Date: May 13, 2004

(54) Antiseptic for wounds and mucous membranes

(76) Inventors: Axel Kramer, Greifswald (DE); Frank-Albert Pitten, Wusterhausen (DE)

Correspondence Address: LERNER, DAVID, LITTENBERG,, KRUMHOLZ & MENTLIK, 600 SOUTH AVENUE WEST, WESTFIELD, NJ, 07090, US

(21) Appl. No.: 10/469,431

(22) PCT Filed: Feb. 27, 2002
(86) PCT No.: PCT/EP02/02108

(30) Foreign Application Priority Data
Mar. 1, 2001 (DE) 101 09 925.8

(51) Int. Cl. A61K 31/205; A61K 31/045

(52) U.S. Cl. 514/554; 514/724

(57) ABSTRACT

The invention relates to an octenidine antiseptic for wounds and mucous membranes that is characterized by a content in ethanol and a physiologically acceptable organic acid.

The present invention concerns a synergistic combination of active ingredients based on octenidine for the antiseptic treatment of mucous membranes and/or wounds.

Octenidine, which has the following formula:

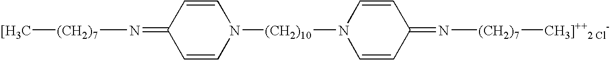

is well known as an antiseptic for mucous membranes and is also used for wound antisepsis, but this compound is ordinarily combined with phenoxyethanol to enhance its effectiveness. For example, a preparation of this description is commercially available under the brand name “Octenisept” and is often used in gynecology and urology. However, recent studies have shown that the combination of octenidine and phenoxyethanol is highly cytotoxic, which raises considerable concerns about its use on open wounds.

Therefore, the goal of the invention is the development of an octenidine-based wound and mucous membrane antiseptic that is stable and significantly less toxic than previously known mixtures.

It is proposed that this problem be solved with a wound and mucous membrane antiseptic that contains octenidine, ethanol, and a physiologically well-tolerated organic acid.

Very surprisingly, it was discovered that a stable mixture of active ingredients with synergistic action can be obtained by combining octenidine with relatively small amounts of ethanol and a very small amount of an organic acid. The mixture preferably contains 0.05 to 0.1 wt. % octenidine, 2-10 wt. % ethanol (94%), and about 0.5 to 2 wt. % of the organic acid.

Organic acids that may be used are hydroxymonocarboxylic acids, such as lactic acid and glycolic acid, dicarboxylic acids, such as malonic acid and succinic acid, and saturated hydroxydicarboxylic and hydroxytricarboxylic acids, such as malic acid, tartaric acid, and citric acid. The pH of the antiseptics of the invention should be in the range of 2.5-3.0, and preferably 2.6, because this range was found to be favorable for use on mucous membranes or wounds.

Surprisingly, the mixtures of the invention are clear, colorless, stable solutions, and they are all extremely stable in storage, which would not have been expected in view of the sparing solubility of octenidine. They are also extremely well tolerated on mucous membranes and open wounds, and it should also be noted that their effectiveness is significantly greater than that of a combination of octenidine and phenoxyethanol or than that of octenidine alone in aqueous solution. They show a true synergistic effect.

The invention is explained in greater detail by the following examples:

### EXAMPLE 1

An aqueous solution containing 0.1% octenidine, 5% ethanol (94%), and 1.5% lactic acid (80%) was tested by the quantitative suspension test. A dilution containing this mixture in a concentration of 25% caused, without loading, a reduction of S. aureus and P. aeruginosa by more than 5 log/units within only 30 seconds. The same result was obtained after it had been allowed to act on C. albicans for 10 minutes. The 90% application concentration of the mixture reduced C. albicans by more than 5 log/units within only 2.5 minutes. With massive albumin and blood loading of 10%, the test bacteria were reduced by more than 4.6 log/units by the 50% application concentration within only 30 seconds, which is a significantly greater reduction than the 3.0 log/units required by the DKH. Under the same loading conditions, adequate reduction of C. albicans was achieved within 4 minutes with the same concentration. Even under a high mucin load of 10%, the 90% application concentration was effective against S. aureus and P. aeruginosa within 30 minutes.

The combination of active ingredients is a product that acts rapidly against bacteria and yeasts. Even under albumin, blood, or mucin loading, it acts rapidly and very effectively.

### EXAMPLE 2

The synergistic mixture of active ingredients is well tolerated in the explant test (A. Kramer et al., Chirurg., Vol. 60, pp. 840-845, 1998). In 10% dilution, an explantation rate of 80% with a growth rate of 60% is achieved. By comparison, when 10% Octenisept is allowed to act for the same amount of time (30 seconds), both explantation and growth are completely suppressed.

This result is especially surprising, because there was no indication that the cytotoxicity should be neutralized by the combination with ethanol and a physiologically acceptable organic acid.

### EXAMPLE 3

In this test, adherent cultures of human amnion cells (FL cells) are incubated for 24 h at 37° C. with the desired concentration of the test substance in complete MEM culture medium with 5% FBS in a 5% CO₂/air atmosphere. The culture medium and test substance are then removed, and new culture medium with neutral red is added. Since vital FL cells take up the dye neutral red in their lysosomes, the intensity of the eluted red coloration is correlated with the proportion of living cells. The intensity is automatically determined by the absorbance at a 540 nm test wavelength/655 nm reference wavelength. Information about the cytotoxic activity of the test substance can be obtained by comparison with a control group that was incubated with phosphate-buffered salt solution (PBS) instead of with the test substance. In addition, this model can be used to test and compare different formulations.

In the present test, different concentrations of octenidine dihydrochloride in PBS and Octenisept® (containing octenidine dihydrochloride and phenoxyethanol) were compared with each other and with PBS. The concentration values refer to the concentration of octenidine dihydrochloride.

### Results

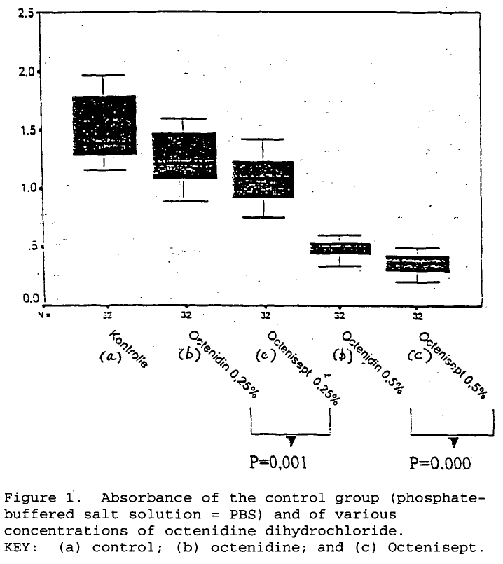

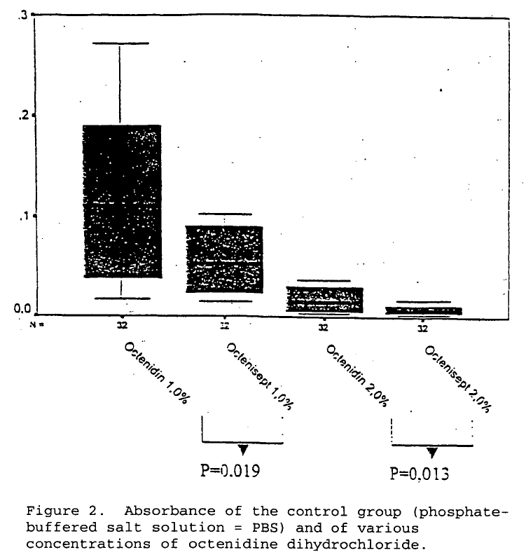

The measured absorbances are plotted in FIGS. 1-2. Each measurement is based on 32 individual measurements. Inferential statistical analysis was performed by the U-test. The tests show that the absorbance of the pure substance octenidine dihydrochloride is significantly elevated relative to that of the commercial preparation Octenisept® at all tested concentrations. This means that the cytotoxic activity of octenidine dihydrochloride is significantly lower than that of comparable concentrations of this active ingredient in the commercial product Octenisept®. The reason for this additive cytotoxic effect can only be the combination of octenidine dihydrochloride in Octenisept® with other substances, most likely phenoxyethanol.

1. Wound and mucous membrane antiseptic based on octenidine, characterized by the fact that it contains ethanol and a physiologically well-tolerated organic acid.

2. Wound and mucous membrane antiseptic in accordance with claim 1, characterized by the fact that it contains hydroxycarboxylic acids, dicarboxylic acids, or hydroxydicarboxylic or hydroxytricarboxylic acids.

3. Wound and mucous membrane antiseptic in accordance with claim 1 or claim 2, characterized by the fact that it contains an acid selected from the group comprising lactic acid, glycolic acid, malonic acid, succinic acid, malic acid, tartaric acid, and citric acid.

4. Wound and mucous membrane antiseptic in accordance with claims 1 to 3 characterized by the fact that it contains 0.05 to 0.1 wt. % octenidine, 2 wt. % ethanol, and 0.5 to 2.5 wt. % of the organic acid.
